SEQUENCE LISTING (56 sequences)

SEQ ID NO: 1 - DNA, 25 bp, synthetic construct
  misc_feature 1..25: note = Forward primer of primer pair 1
  source 1..25: mol_type = other DNA
tcacttgactaatcgtgtcatgaca

SEQ ID NO: 2 - DNA, 25 bp, synthetic construct
  misc_feature 1..25: note = Reverse primer of primer pair 1
  source 1..25: mol_type = other DNA
agaatggactaacgttgagaggatt

SEQ ID NO: 3 - DNA, 25 bp, synthetic construct
  misc_feature 1..25: note = Forward primer of primer pair 2
  source 1..25: mol_type = other DNA
gctaacccgctctatgttaaagttc

SEQ ID NO: 4 - DNA, 25 bp, synthetic construct
  misc_feature 1..25: note = Reverse primer of primer pair 2
  source 1..25: mol_type = other DNA
atagagacagagccaactcaaactt

SEQ ID NO: 5 - DNA, 25 bp, synthetic construct
  misc_feature 1..25: note = Forward primer of primer pair 3
  source 1..25: mol_type = other DNA
aacaccaaactatcactaggtttgc

SEQ ID NO: 6 - DNA, 25 bp, synthetic construct
  misc_feature 1..25: note = Reverse primer of primer pair 3
  source 1..25: mol_type = other DNA
ccaggtcacttacaccttaacacta

SEQ ID NO: 7 - DNA, 25 bp, synthetic construct
  misc_feature 1..25: note = Forward primer of primer pair 4
  source 1..25: mol_type = other DNA
gatgtctgcctctatggtagcttaa

SEQ ID NO: 8 - DNA, 25 bp, synthetic construct
  misc_feature 1..25: note = Reverse primer of primer pair 4
  source 1..25: mol_type = other DNA
agtagttgtaagcataccaccatga

SEQ ID NO: 9 - DNA, 25 bp, synthetic construct
  misc_feature 1..25: note = Forward primer of primer pair 5
  source 1..25: mol_type = other DNA
gcaatcaatttgtgtctttatgccc

SEQ ID NO: 10 - DNA, 25 bp, synthetic construct
  misc_feature 1..25: note = Reverse primer of primer pair 5
  source 1..25: mol_type = other DNA
ttttgaatcctagacatgcatgcaa

SEQ ID NO: 11 - DNA, 25 bp, synthetic construct
  misc_feature 1..25: note = Forward primer of primer pair 6
  source 1..25: mol_type = other DNA
tcaccatttagattcttctcaggca

SEQ ID NO: 12 - DNA, 25 bp, synthetic construct
  misc_feature 1..25: note = Reverse primer of primer pair 6
  source 1..25: mol_type = other DNA
ttcgtgagctattgtttttcatcgt

SEQ ID NO: 13 - DNA, 25 bp, synthetic construct
  misc_feature 1..25: note = Forward primer of primer pair 7
  source 1..25: mol_type = other DNA
agtcatgtcagcaattcagcttatg

SEQ ID NO: 14 - DNA, 25 bp, synthetic construct
  misc_feature 1..25: note = Reverse primer of primer pair 7
  source 1..25: mol_type = other DNA
gtctccctctctttcatttctacga

SEQ ID NO: 15 - DNA, 25 bp, synthetic construct
  misc_feature 1..25: note = Forward primer of primer pair 8
  source 1..25: mol_type = other DNA
tcaaattttaacctcagaggacagg

SEQ ID NO: 16 - DNA, 31 bp, synthetic construct
  misc_feature 1..31: note = Reverse primer of primer pair 8
  source 1..31: mol_type = other DNA
tgacaatgaagataatgataacaaaattagt

SEQ ID NO: 17 - DNA, 25 bp, synthetic construct
  misc_feature 1..25: note = Forward primer of primer pair 9
  source 1..25: mol_type = other DNA
atcatagtcaggggtggacctatat

SEQ ID NO: 18 - DNA, 25 bp, synthetic construct
  misc_feature 1..25: note = Reverse primer of primer pair 9
  source 1..25: mol_type = other DNA
aacaacatcattctgatcagtggtg

SEQ ID NO: 19 - DNA, 25 bp, synthetic construct
  misc_feature 1..25: note = Reverse primer of primer pair 10
  source 1..25: mol_type = other DNA
gacaccaatcaaaaattgaactgca

SEQ ID NO: 20 - DNA, 25 bp, synthetic construct
  misc_feature 1..25: note = Forward primer of primer pair 10
  source 1..25: mol_type = other DNA
actcaaccaataaaagagcatgcaa

SEQ ID NO: 21 - DNA, 25 bp, synthetic construct
  misc_feature 1..25: note = Forward primer of primer pair 11
  source 1..25: mol_type = other DNA
tcaagcactttgatttccattctgt

SEQ ID NO: 22 - DNA, 26 bp, synthetic construct
  misc_feature 1..26: note = Reverse primer of primer pair 11
  source 1..26: mol_type = other DNA
cgtgtagtatagttttgaaaatggcg

SEQ ID NO: 23 - DNA, 25 bp, synthetic construct
  misc_feature 1..25: note = Forward primer of primer pair 12
  source 1..25: mol_type = other DNA
aaaaaccatccttacaaaactgcca

SEQ ID NO: 24 - DNA, 26 bp, synthetic construct
  misc_feature 1..26: note = Reverse primer of primer pair 12
  source 1..26: mol_type = other DNA
tcagtgggatctgattgtattttacc

SEQ ID NO: 25 - DNA, 25 bp, synthetic construct
  misc_feature 1..25: note = Forward primer of primer pair 13
  source 1..25: mol_type = other DNA
aaaacatctcttcatgctgttgtcc

SEQ ID NO: 26 - DNA, 25 bp, synthetic construct
  misc_feature 1..25: note = Reverse primer of primer pair 13
  source 1..25: mol_type = other DNA
aaacctaactaagcttcggtctcaa

SEQ ID NO: 27 - DNA, 29 bp, synthetic construct
  misc_feature 1..29: note = Forward primer of primer pair 14
  source 1..29: mol_type = other DNA
tgtatacacgaaagatggaatattctttt

SEQ ID NO: 28 - DNA, 25 bp, synthetic construct
  misc_feature 1..25: note = Reverse primer of primer pair 14
  source 1..25: mol_type = other DNA
tgccggaaaattttatggcatagat

SEQ ID NO: 29 - DNA, 25 bp, synthetic construct
  misc_feature 1..25: note = Forward primer of primer pair 15
  source 1..25: mol_type = other DNA
ttaagcagttcctctcatcacgtaa

SEQ ID NO: 30 - DNA, 25 bp, synthetic construct
  misc_feature 1..25: note = Reverse primer of primer pair 15
  source 1..25: mol_type = other DNA
tggatagttgactttgtttggttgg

SEQ ID NO: 31 - DNA, 25 bp, synthetic construct
  misc_feature 1..25: note = Forward primer of primer pair 16
  source 1..25: mol_type = other DNA
agttggttaagtcgattaggcttga

SEQ ID NO: 32 - DNA, 25 bp, synthetic construct
  misc_feature 1..25: note = Reverse primer of primer pair 16
  source 1..25: mol_type = other DNA
gccaaaaatgagcaaaaatgcaaca

SEQ ID NO: 33 - DNA, 25 bp, synthetic construct
  misc_feature 1..25: note = Forward primer of primer pair 17
  source 1..25: mol_type = other DNA
gagctattcctttgattgaaaccca

SEQ ID NO: 34 - DNA, 25 bp, synthetic construct
  misc_feature 1..25: note = Reverse primer of primer pair 17
  source 1..25: mol_type = other DNA
ttgggagcttatactgaggtttctt

SEQ ID NO: 35 - DNA, 26 bp, synthetic construct
  misc_feature 1..26: note = Forward primer of primer pair 18
  source 1..26: mol_type = other DNA
tgtttagtcaattcaggtcgataagt

SEQ ID NO: 36 - DNA, 25 bp, synthetic construct
  misc_feature 1..25: note = Reverse primer of primer pair 18
  source 1..25: mol_type = other DNA
gttcacatgcttgtaccgattgata

SEQ ID NO: 37 - DNA, 25 bp, synthetic construct
  misc_feature 1..25: note = Forward primer of primer pair 19
  source 1..25: mol_type = other DNA
ccagagttaacatctcggtttgatg

SEQ ID NO: 38 - DNA, 25 bp, synthetic construct
  misc_feature 1..25: note = Reverse primer of primer pair 19
  source 1..25: mol_type = other DNA
aatttggccaaattcaaactggtca

SEQ ID NO: 39 - DNA, 25 bp, synthetic construct
  misc_feature 1..25: note = Forward primer of primer pair 20
  source 1..25: mol_type = other DNA
ttttagccaaagaacatgttatggt

SEQ ID NO: 40 - DNA, 28 bp, synthetic construct
  misc_feature 1..28: note = Reverse primer of primer pair 20
  source 1..28: mol_type = other DNA
acttttattcaacatgcttttcataagt

SEQ ID NO: 41 - DNA, 25 bp, synthetic construct
  misc_feature 1..25: note = Forward primer of primer pair 21
  source 1..25: mol_type = other DNA
cgcccaccaatagaaataattgtga

SEQ ID NO: 42 - DNA, 28 bp, synthetic construct
  misc_feature 1..28: note = Reverse primer of primer pair 21
  source 1..28: mol_type = other DNA
tgtgatcggatgttaattagctttttca

SEQ ID NO: 43 - DNA, 25 bp, synthetic construct
  misc_feature 1..25: note = Forward primer of primer pair 22
  source 1..25: mol_type = other DNA
tatccatcgtgttgctgtcatactt

SEQ ID NO: 44 - DNA, 29 bp, synthetic construct
  misc_feature 1..29: note = Reverse primer of primer pair 22
  source 1..29: mol_type = other DNA
accattgtccttatcatattcgttaaaaa

SEQ ID NO: 45 - DNA, 25 bp, synthetic construct
  misc_feature 1..25: note = Forward primer of primer pair 23
  source 1..25: mol_type = other DNA
taattctagctggcctttagaacgt

SEQ ID NO: 46 - DNA, 25 bp, synthetic construct
  misc_feature 1..25: note = Reverse primer of primer pair 23
  source 1..25: mol_type = other DNA
atatttaagtggcgttggattcgac

SEQ ID NO: 47 - DNA, 25 bp, synthetic construct
  misc_feature 1..25: note = Forward primer of primer pair 24
  source 1..25: mol_type = other DNA
gcacacatcatttctttgtttgacc

SEQ ID NO: 48 - DNA, 25 bp, synthetic construct
  misc_feature 1..25: note = Reverse primer of primer pair 24
  source 1..25: mol_type = other DNA
tgtgttccgattttgattgcgataa

SEQ ID NO: 49 - DNA, 25 bp, synthetic construct
  misc_feature 1..25: note = Forward primer of primer pair 25
  source 1..25: mol_type = other DNA
tctttgtaagatcacgccattattt

SEQ ID NO: 50 - DNA, 25 bp, synthetic construct
  misc_feature 1..25: note = Reverse primer of primer pair 25
  source 1..25: mol_type = other DNA
gttgcattgcacattaggttttctg

SEQ ID NO: 51 - DNA, 31 bp, synthetic construct
  misc_feature 1..31: note = Forward primer of primer pair 26
  source 1..31: mol_type = other DNA
acaaaaatcaacaacgttaacaatataaatt

SEQ ID NO: 52 - DNA, 22 bp, synthetic construct
  misc_feature 1..22: note = Reverse primer of primer pair 26
  source 1..22: mol_type = other DNA
ggcagggtggaagtgaattttt

SEQ ID NO: 53 - DNA, 25 bp, synthetic construct
  misc_feature 1..25: note = Forward primer of primer pair 27
  source 1..25: mol_type = other DNA
tacgtattttcattgcacaagttgt

SEQ ID NO: 54 - DNA, 29 bp, synthetic construct
  misc_feature 1..29: note = Reverse primer of primer pair 27
  source 1..29: mol_type = other DNA
tgtctatgttctaccattaaatgatgaca

SEQ ID NO: 55 - DNA, 25 bp, synthetic construct
  misc_feature 1..25: note = Forward primer of primer pair 28
  source 1..25: mol_type = other DNA
atccccaaagttatggaagagtcaa

SEQ ID NO: 56 - DNA, 25 bp, synthetic construct
  misc_feature 1..25: note = Reverse primer of primer pair 28
  source 1..25: mol_type = other DNA
tcaagaaacaaaaggtatgcatgct

(19) United States
(12) Patent Application Publication
(10) Pub. No.: US 2026/0043092 A1
(43) Pub. Date: Feb. 12, 2026

(54) Method for High-Throughput Multiplex PCR in Soybean Using Simple Sequence Repeat Marker

(71) Applicant: Institute of Crop Sciences, Chinese Academy of Agricultural Sciences, Beijing (CN)

(72) Inventors: Lijuan QIU, Beijing (CN); Rongxia GUAN, Beijing (CN); Xiaoyang GUO, Beijing (CN); Jiaming ZHANG, Beijing (CN)

(21) Appl. No.: 19/222,999

(22) Filed: May 29, 2025

(30) Foreign Application Priority Data
Aug. 7, 2024 (CN) 202411078139.8

(51) Int. Cl. C12Q 1/6895; C12Q 1/686

(52) U.S. Cl. CPC C12Q 1/6895; C12Q 1/686; C12Q 2600/156; C12Q 2600/16

(57) ABSTRACT

A method for high-throughput multiplex PCR in soybean using a simple sequence repeat (SSR) marker is provided, relating to the technical field of molecular biology. 28 pairs of SSR primers that can be used for multiplex PCR amplification and capillary electrophoresis detection are screened out of 38 pairs of SSR primers, in which 28 SSR markers can be divided into 3 groups. Compared with traditional SSR marker detection, 9-10 pairs of primers in the developed SSR markers can complete PCR amplification and capillary electrophoresis in one well, greatly reducing experimental cost and improving detection efficiency. Different primers are labeled with different fluorescent groups, and a length of an amplified fragment at each SSR site for each soybean material is obtained by the capillary electrophoresis.

### CROSS-REFERENCE TO RELATED APPLICATION

This patent application claims the benefit and priority of Chinese Patent Application No. 2024110781398 filed with the China National Intellectual Property Administration on Aug. 7, 2024, the disclosure of which is incorporated by reference herein in its entirety as part of the present application.

### REFERENCE TO SEQUENCE LISTING

A computer readable XML file entitled “GWP20250200382-sequence listing.xml”, which was created on Mar. 4, 2025, with a file size of about 72,760 bytes, contains the sequence listing for this application, has been filed with this application, and is hereby incorporated by reference in its entirety.

### TECHNICAL FIELD

The present disclosure relates to the technical field of molecular biology, and more specifically to a method for high-throughput multiplex PCR in a soybean using a simple sequence repeat (SSR) marker.

### BACKGROUND

Simple sequence repeat (SSR) markers exhibit the advantages of rich variation, simple operation, reliable results, and desirable repeatability. With the development of molecular biology, the SSR markers have been widely used in the identification of germplasm resources, analysis of genetic diversity, detection of variety purity and authenticity, mining of quantitative trait loci (QTLs)/genes, and construction of variety DNA fingerprint database.

Currently, only the genotype information of a single locus can be obtained by analyze every single SSR primer, resulting in low throughput of data analysis. Establishing a multiplex SSR detection system is an important way to increase the amount of information in each SSR analysis. In the multiplex SSR detection system, PCR amplification on multiple pairs of primers is conducted simultaneously while resulting amplified products are detected in a same capillary tube, showing high throughput and less reagents required. However, there are few studies on the multiplex SSR marker detection system in soybean.

Therefore, it is of great significance to establish a high-throughput automatic detection platform and to improve the efficiency of SSR marker detection to construct a complex detection system for the multiplex PCR product based on a capillary four-color fluorescence detection system.

### SUMMARY

In view of this, an objective of the present disclosure is to provide a method for high-throughput multiplex PCR in soybean using an SSR marker.

To achieve the above objective, the present disclosure adopts the following technical solutions:

The present disclosure provides a method for high-throughput multiplex PCR in soybean using an SSR marker, including the following sequences of an SSR primer set:

primer pair 1:
TCACTTGACTAATCGTGTCATGACA set forth in SEQ ID NO: 1;
AGAATGGACTAACGTTGAGAGGATT set forth in SEQ ID NO: 2;
primer pair 2:
GCTAACCCGCTCTATGTTAAAGTTC set forth in SEQ ID NO: 3;
ATAGAGACAGAGCCAACTCAAACTT set forth in SEQ ID NO: 4;
primer pair 3:
AACACCAAACTATCACTAGGTTTGC set forth in SEQ ID NO: 5;
CCAGGTCACTTACACCTTAACACTA set forth in SEQ ID NO: 6;
primer pair 4:
GATGTCTGCCTCTATGGTAGCTTAA set forth in SEQ ID NO: 7;
AGTAGTTGTAAGCATACCACCATGA set forth in SEQ ID NO: 8;
primer pair 5:
GCAATCAATTTGTGTCTTTATGCCC set forth in SEQ ID NO: 9;
TTTTGAATCCTAGACATGCATGCAA set forth in SEQ ID NO: 10;
primer pair 6:
TCACCATTTAGATTCTTCTCAGGCA set forth in SEQ ID NO: 11;
TTCGTGAGCTATTGTTTTTCATCGT set forth in SEQ ID NO: 12;
primer pair 7:
AGTCATGTCAGCAATTCAGCTTATG set forth in SEQ ID NO: 13;
GTCTCCCTCTCTTTCATTTCTACGA set forth in SEQ ID NO: 14;
primer pair 8:
TCAAATTTTAACCTCAGAGGACAGG set forth in SEQ ID NO: 15;
TGACAATGAAGATAATGATAACAAAATTAGT set forth in SEQ ID NO: 16;
primer pair 9:
ATCATAGTCAGGGGTGGACCTATAT set forth in SEQ ID NO: 17;
AACAACATCATTCTGATCAGTGGTG set forth in SEQ ID NO: 18;
primer pair 10:
GACACCAATCAAAAATTGAACTGCA set forth in SEQ ID NO: 19;
ACTCAACCAATAAAAGAGCATGCAA set forth in SEQ ID NO: 20;
primer pair 11:
TCAAGCACTTTGATTTCCATTCTGT set forth in SEQ ID NO: 21;
CGTGTAGTATAGTTTTGAAAATGGCG set forth in SEQ ID NO: 22;
primer pair 12:
AAAAACCATCCTTACAAAACTGCCA set forth in SEQ ID NO: 23;
TCAGTGGGATCTGATTGTATTTTACC set forth in SEQ ID NO: 24;
primer pair 13:
AAAACATCTCTTCATGCTGTTGTCC set forth in SEQ ID NO: 25;
AAACCTAACTAAGCTTCGGTCTCAA set forth in SEQ ID NO: 26;
primer pair 14:
TGTATACACGAAAGATGGAATATTCTTTT set forth in SEQ ID NO: 27;
TGCCGGAAAATTTTATGGCATAGAT set forth in SEQ ID NO: 28;
primer pair 15:
TTAAGCAGTTCCTCTCATCACGTAA set forth in SEQ ID NO: 29;
TGGATAGTTGACTTTGTTTGGTTGG set forth in SEQ ID NO: 30;
primer pair 16:
AGTTGGTTAAGTCGATTAGGCTTGA set forth in SEQ ID NO: 31;
GCCAAAAATGAGCAAAAATGCAACA set forth in SEQ ID NO: 32;
primer pair 17:
GAGCTATTCCTTTGATTGAAACCCA set forth in SEQ ID NO: 33;
TTGGGAGCTTATACTGAGGTTTCTT set forth in SEQ ID NO: 34;
primer pair 18:
TGTTTAGTCAATTCAGGTCGATAAGT set forth in SEQ ID NO: 35;
GTTCACATGCTTGTACCGATTGATA set forth in SEQ ID NO: 36;
primer pair 19:
CCAGAGTTAACATCTCGGTTTGATG set forth in SEQ ID NO: 37;
AATTTGGCCAAATTCAAACTGGTCA set forth in SEQ ID NO: 38;
primer pair 20:
TTTTAGCCAAAGAACATGTTATGGT set forth in SEQ ID NO: 39;
ACTTTTATTCAACATGCTTTTCATAAGT set forth in SEQ ID NO: 40;
primer pair 21:
CGCCCACCAATAGAAATAATTGTGA set forth in SEQ ID NO: 41;
TGTGATCGGATGTTAATTAGCTTTTTCA set forth in SEQ ID NO: 42;
primer pair 22:
TATCCATCGTGTTGCTGTCATACTT set forth in SEQ ID NO: 43;
ACCATTGTCCTTATCATATTCGTTAAAAA set forth in SEQ ID NO: 44;
primer pair 23:
TAATTCTAGCTGGCCTTTAGAACGT set forth in SEQ ID NO: 45;
ATATTTAAGTGGCGTTGGATTCGAC set forth in SEQ ID NO: 46;
primer pair 24:
GCACACATCATTTCTTTGTTTGACC set forth in SEQ ID NO: 47;
TGTGTTCCGATTTTGATTGCGATAA set forth in SEQ ID NO: 48;
primer pair 25:
TCTTTGTAAGATCACGCCATTATTT set forth in SEQ ID NO: 49;
GTTGCATTGCACATTAGGTTTTCTG set forth in SEQ ID NO: 50;
primer pair 26:
ACAAAAATCAACAACGTTAACAATATAAATT set forth in SEQ ID NO: 51;
GGCAGGGTGGAAGTGAATTTTT set forth in SEQ ID NO: 52;
primer pair 27:
TACGTATTTTCATTGCACAAGTTGT set forth in SEQ ID NO: 53;
TGTCTATGTTCTACCATTAAATGATGACA set forth in SEQ ID NO: 54;
primer pair 28:
ATCCCCAAAGTTATGGAAGAGTCAA set forth in SEQ ID NO: 55;
TCAAGAAACAAAAGGTATGCATGCT set forth in SEQ ID NO: 56.

Further, the method for high-throughput multiplex PCR in soybean using an SSR marker includes the following steps:
(1) extracting DNA from a soybean sample;
(2) establishing a PCR reaction system using the DNA as a template and the SSR primer set according to claim 1 as an amplification primer and conducting PCR amplification; and
(3) subjecting an obtained amplified product to capillary electrophoresis, and then detecting a length of an obtained fragment.

Further, the PCR reaction system for the PCR amplification is 25 μL and includes 1.5 μL of primer mixture, 5 μL of DNA, 12.5 μL of Premix Ex Taq Hot Start enzyme (RR030A, Takara), and 6 μL of water;
a single primer in the primer mixture has a concentration of 0.4 μM, and the DNA has a concentration of 20 ng/μL; and
a procedure of the PCR amplification includes: initial denaturation at 95° C. for 3 min; 32 cycles for a process of denaturation at 95° C. for 30 s and then annealing at 60° C. for 4 min; and extension at 72° C. for 5 min.

Further, a soybean variety to be identified includes:

Xingnong 25, Suinong 117, Zhonglong 606, Longken 397, Jiyu 209, Xingnong 9, Henong 81, Kendou 94, Fenghedou 305, Tiedou 102, Liaodou 69, Tiedou 110, Shengdou 21, Zhonghuang 203, Zhonghuang 212, Andou 6223, Jidou 29, Zhonghuang 211, Ji 1901, Andou 6263, Handou 13, Shanning 29, Ji 1801, Pudou 754. Heyu 10hao, Liudou 108, Huaidou 17, Nannong 60, Shengyu 6hao, Xu 9416-8, Hedou 37, Zhongdou 48, Zhongdou 66, Xiangchun 2704, Wandou 40, Huadou 15, Zhoudou 49, Huadou 16, Wandou 61, Jiaoda 23, Jiaoda 28, Zhejiangxian 86, Huachun 12, Sudou 051, Zhoudou 38, Zhongdou 6301, Wansu 061, and Wansu 112. These varieties can be queried and available from a Chinese seed supplier, website: https://www.chinasced114.com/.

It can be seen from the above technical solutions that compared with the prior art, the present disclosure has the following beneficial effects:

Twenty-eight pairs of SSR primers that can be used for multiplex PCR amplification and capillary electrophoresis detection are screened out of 38 pairs of SSR primers, in which 28 SSR markers can be divided into 3 groups. Compared with traditional SSR marker detection, 9-10 pairs of primers in the developed SSR markers can complete PCR amplification and capillary electrophoresis in one well, greatly reducing experimental cost and improving detection efficiency. Different primers are labeled with different fluorescent groups, and a length of an amplified fragment at each SSR site for each soybean material can be obtained by the capillary electrophoresis.

### DETAILED DESCRIPTION OF THE EMBODIMENTS

The technical solutions in the embodiments of the present disclosure will be clearly and completely described below. Apparently, the described embodiments are merely a part of, not all of, the embodiments of the present disclosure. All other examples obtained by those skilled in the art based on the examples of the present disclosure without creative efforts shall fall within the protection scope of the present disclosure.

### Example 1

1) Redesign of primers and establishment of universal PCR program: The 38 pairs of primers were redesigned according to unified requirements such that the amplification conditions of the primers were similar and the size of the amplification products was within the expected range, which was convenient for the subsequent combination of primers. After trial and error, the annealing temperature of the new primer was determined to be 60° C., and a two-step amplification program was established: initial denaturation at 95° C. for 3 min; 32 cycles for a process of denaturation at 95° C. for 30 s and then annealing at 60° C. for 4 min; and extension at 72° C. for 5 min.

The original primers and the new primers were used uniformly in the above PCR program to amplify 12 soybean varieties, and the amplification band patterns and amplification effects of the two primers were compared. The primers that had poor amplification effect or did not match the expected band size were redesigned. Finally, 28 pairs of candidate primers for multiplex PCR product detection were determined.
2) Establishment of multiplex detection system of fluorescent primer PCR products: According to the above amplification results, 28 pairs of primers were combined into 3 primer-mixing schemes based on amplification efficiency, amplification product size, and fluorescent group. The primer groups were determined according to the molecular weight and fluorescent group of the primers. Primer pairs 1 to 10 were Group 1, primer pairs 11 to 19 were Group 2, and primer pairs 20 to 28 were Group 3, and the primer information is shown in Table 1.

The final soybean multiplex PCR amplification system (25 μL) included: 1.5 μL of primers (concentration of each primer 0.4 μM), 5 μL of DNA (20 ng/μL), 12.5 μL of Premix Ex Taq Hot Start enzyme (RR030A, Takara), and 6 μL of water. The amplification program of SSR multiplex PCR was as follows: initial denaturation at 95° C. for 3 min; 32 cycles for a process of denaturation at 95° C. for 30 s and then annealing at 60° C. for 4 min; and extension at 72° C. for 5 min.

TABLE 1
Primer information

| | | Molecular | | | | | |
|---|---|---|---|---|---|---|---|
| | | weight | Fluor- | | | | |
| Primer | | range | escent | | | | |
| pair | Code | (bp) | group | Forward primer | SN | Reverse primer | SN |
| 1 | Gm029 | 329-357 | NED | TCACTTGACTAATCG | 1 | AGAATGGACTAACGTT | 2 |
| | | | | TGTCATGACA | | GAGAGGATT | |
| 2 | Gm010 | 172-180 | FAM | GCTAACCCGCTCTAT | 3 | ATAGAGACAGAGCCA | 4 |
| | | | | GTTAAAGTTC | | ACTCAAACTT | |
| 3 | Gm001 | 253-282 | FAM | AACACCAAACTATC | 5 | CCAGGTCACTTACACC | 6 |
| | | | | ACTAGGTTTGC | | TTAACACTA | |
| 4 | Gm070 | 317-362 | FAM | GATGTCTGCCTCTAT | 7 | AGTAGTTGTAAGCATA | 8 |
| | | | | GGTAGCTTAA | | CCACCATGA | |
| 5 | Gm056 | 260-287 | VIC | GCAATCAATTTGTGT | 9 | TTTTGAATCCTAGACA | 10 |
| | | | | CTTTATGCCC | | TGCATGCAA | |
| 6 | Gm012 | 299-311 | NED | TCACCATTTAGATTC | 11 | TTCGTGAGCTATTGTT | 12 |
| | | | | TTCTCAGGCA | | TTTCATCGT | |
| 7 | Gm007 | 223-262 | NED | AGTCATGTCAGCAA | 13 | GTCTCCCTCTCTTTCAT | 14 |
| | | | | TTCAGCTTATG | | TTCTACGA | |
| 8 | Gm033 | 131-156 | NED | TCAAATTTTAACCTC | 15 | TGACAATGAAGATAAT | 16 |
| | | | | AGAGGACAGG | | GATAACAAAATTAGT | |
| 9 | Gm027 | 231-277 | PET | ATCATAGTCAGGGG | 17 | AACAACATCATTCTGA | 18 |
| | | | | TGGACCTATAT | | TCAGTGGTG | |
| 10 | Gm022 | 136-151 | PET | GACACCAATCAAAA | 19 | ACTCAACCAATAAAA | 20 |
| | | | | ATTGAACTGCA | | GAGCATGCAA | |
| 11 | Gm043 | 85-105 | FAM | TCAAGCACTTTGATT | 21 | CGTGTAGTATAGTTTT | 22 |
| | | | | TCCATTCTGT | | GAAAATGGCG | |
| 12 | Gm004 | 189-211 | FAM | AAAAACCATCCTTA | 23 | TCAGTGGGATCTGATT | 24 |
| | | | | CAAAACTGCCA | | GTATTTTACC | |
| 13 | Gm065 | 280-306 | FAM | AAAACATCTCTTCAT | 25 | AAACCTAACTAAGCTT | 26 |
| | | | | GCTGTTGTCC | | CGGTCTCAA | |
| 14 | Gm051 | 125-167 | VIC | TGTATACACGAAAG | 27 | TGCCGGAAAATTTTAT | 28 |
| | | | | ATGGAATATTCTTTT | | GGCATAGAT | |
| 15 | Gm054 | 239-280 | VIC | TTAAGCAGTTCCTCT | 29 | TGGATAGTTGACTTTG | 30 |
| | | | | CATCACGTAA | | TTTGGTTGG | |
| 16 | Gm045 | 320-383 | VIC | AGTTGGTTAAGTCG | 31 | GCCAAAAATGAGCAA | 32 |
| | | | | ATTAGGCTTGA | | AAATGCAACA | |
| 17 | Gm006 | 85-139 | NED | GAGCTATTCCTTTGA | 33 | TTGGGAGCTTATACTG | 34 |
| | | | | TTGAAACCCA | | AGGTTTCTT | |
| 18 | Gm053 | 329-356 | NED | TGTTTAGTCAATTCA | 35 | GTTCACATGCTTGTAC | 36 |
| | | | | GGTCGATAAGT | | CGATTGATA | |
| 19 | Gm046 | 167-220 | PET | CCAGAGTTAACATC | 37 | AATTTGGCCAAATTCA | 38 |
| | | | | TCGGTTTGATG | | AACTGGTCA | |
| 20 | Gm055 | 124-162 | FAM | TTTTAGCCAAAGAA | 39 | ACTTTTATTCAACATG | 40 |
| | | | | CATGTTATGGT | | CTTTTCATAAGT | |
| 21 | Gm049 | 214-246 | FAM | CGCCCACCAATAGA | 41 | TGTGATCGGATGTTAA | 42 |
| | | | | AATAATTGTGA | | TTAGCTTTTTCA | |
| 22 | Gm059 | 271-309 | FAM | TATCCATCGTGTTGC | 43 | ACCATTGTCCTTATCA | 44 |
| | | | | TGTCATACTT | | TATTCGTTAAAAA | |
| 23 | Gm052 | 351-382 | FAM | TAATTCTAGCTGGCC | 45 | ATATTTAAGTGGCGTT | 46 |
| | | | | TTTAGAACGT | | GGATTCGAC | |
| 24 | Gm005 | 302-337 | VIC | GCACACATCATTTCT | 47 | TGTGTTCCGATTTTGA | 48 |
| | | | | TTGTTTGACC | | TTGCGATAA | |
| 25 | Gm019 | 198-240 | VIC | TCTTTGTAAGATCAC | 49 | GTTGCATTGCACATTA | 50 |
| | | | | GCCATTATTT | | GGTTTTCTG | |
| 26 | Gm014 | 107-149 | NED | CGTTAACAATATAA | 51 | GGCAGGGTGGAAGTG | 52 |
| | | | | ACAAAAATCAACAA | | AATTTTT | |
| | | | | ATT | | | |
| 27 | Gm048 | 155-179 | PET | TACGTATTTTCATTG | 53 | TGTCTATGTTCTACCA | 54 |
| | | | | CACAAGTTGT | | TTAAATGATGACA | |
| 28 | Gm032 | 299-329 | PET | ATCCCCAAAGTTAT | 55 | TCAAGAAACAAAAGG | 56 |
| | | | | GGAAGAGTCAA | | TATGCATGCT | |

### Multiplex PCR of 48 Soybean Varieties (Lines)

### (1) Extraction of Genomic DNA from Soybean Varieties

Forty-eight soybean varieties (lines) were selected, and 0.1 g of young leaves were taken from each soybean. Soybean genomic DNA was extracted using a DNA extraction kit (Genomic DNA Purification Kit, #K0512, Thermo Fisher Scientific), diluted to 20 ng/μL, and stored in a −20° C. refrigerator for future use.

### (2) PCR Amplification and Capillary Electrophoresis Detection

The PCR amplification system was 25 μL in volume, including 1.5 μL of primer mixture (mixed the primers separately to obtain Groups 1 to 3, each primer concentration was 0.4 μM), 5 μL of DNA (20 ng/μL), 12.5 μL of Premix Ex Taq Hot Start enzyme (RR030A, Takara), and 6 μL of water; where a procedure of the PCR amplification included: initial denaturation at 95° C. for 3 min; 32 cycles for a process of denaturation at 95° C. for 30 s and then annealing at 60° C. for 4 min; and extension at 72° C. for 5 min. After the amplification was completed, the products amplified by primer Groups 1 to 3 were subjected to capillary electrophoresis using an ABI3730 high-throughput gene analyzer to detect the fragment length (bp).

### (3) Analysis of Multiplex PCR Results of 48 Soybean Varieties (Lines)

The capillary electrophoresis results were standardized. If only 1 band was amplified for each site, it was recorded as homozygous, such as 180/180; if 2 or more bands were amplified at the same time, it was recorded as heterozygous, such as 180/183. The results are shown in Table 2.

TABLE 2
Results of 28 SSR markers amplified in 48 soybean varieties (lines)

| Variety name | Gm001 | Gm004 | Gm005 | Gm006 | Gm007 | Gm010 | Gm012 | Gm014 | Gm019 |
|---|---|---|---|---|---|---|---|---|---|
| Xingnong 25 | 282/282 | 200/200 | 330/330 | 131/131 | 246/246 | 182/182 | 306/306 | 110/110 | 216/216 |
| Suinong 117 | 282/282 | 200/200 | 302/302 | 140/140 | 246/246 | 182/182 | 300/300 | 123/123 | 204/204 |
| Zhongnong 606 | 265/265 | 209/209 | 330/330 | 131/131 | 243/249 | 179/182 | 300/312 | 123/123 | 236/236 |
| Longken 397 | 265/265 | 200/200 | 330/330 | 131/131 | 246/246 | 182/182 | 300/300 | 110/110 | 204/204 |
| Jiyu 209 | 265/265 | 209/209 | 302/302 | 131/131 | 243/243 | 179/179 | 300/300 | 145/145 | 204/204 |
| Xingnong 9 | 265/265 | 188/188 | 330/330 | 131/131 | 246/246 | 182/182 | 312/312 | 113/113 | 216/216 |
| Henong 81 | 282/282 | 200/200 | 330/330 | 131/131 | 246/246 | 182/182 | 306/306 | 110/123 | 216/216 |
| Kendou 94 | 265/265 | 188/188 | 330/330 | 131/131 | 246/246 | 182/182 | 300/300 | 145/145 | 216/216 |
| Fenghedou 305 | 265/265 | 200/200 | 330/330 | 131/131 | 246/246 | 182/182 | 300/300 | 148/148 | 204/204 |
| Tiedou 102 | 265/265 | 200/200 | 330/330 | 116/116 | 243/243 | 179/179 | 312/312 | 148/148 | 204/204 |
| Liaodou 69 | 265/265 | 209/209 | 330/330 | 116/116 | 243/243 | 179/179 | 306/306 | 107/107 | 204/204 |
| Tiedou 110 | 265/265 | 188/188 | 330/330 | 116/116 | 243/243 | 179/179 | 306/306 | 129/129 | 204/204 |
| Shengdou 21 | 274/274 | 209/209 | 317/317 | 137/137 | 249/249 | 176/176 | 306/306 | 126/126 | 204/204 |
| Zhonghuang 203 | 282/282 | 209/209 | 327/327 | 131/131 | 243/243 | 176/176 | 312/312 | 148/148 | 201/201 |
| Zhonghuang 212 | 282/282 | 200/200 | 327/327 | 140/140 | 243/243 | 176/176 | 312/312 | 126/126 | 201/201 |
| Andou 6223 | 274/274 | 209/209 | 324/324 | 137/137 | 252/252 | 179/179 | 300/300 | 129/129 | 204/204 |
| Jidou 29 | 282/282 | 200/200 | 333/333 | 137/137 | 243/243 | 176/176 | 312/312 | 129/129 | 204/204 |
| Zhonghuang 211 | 282/282 | 209/209 | 327/327 | 131/131 | 243/243 | 176/176 | 312/312 | 148/148 | 201/201 |
| Ji 1901 | 282/282 | 200/200 | 333/333 | 116/116 | 243/243 | 179/179 | 312/312 | 148/148 | 204/204 |
| Andou 6263 | 274/274 | 206/206 | 324/324 | 137/137 | 246/246 | 179/179 | 300/300 | 113/113 | 204/204 |
| Handou 13 | 274/274 | 206/206 | 302/302 | 150/150 | 243/243 | 179/179 | 306/306 | 126/126 | 204/204 |
| Shanning 29 | 274/274 | 209/209 | 314/314 | 86/140 | 246/246 | 179/179 | 312/312 | 107/107 | 204/204 |
| Ji 1801 | 274/274 | 209/209 | 330/330 | 137/137 | 243/243 | 179/179 | 312/312 | 110/110 | 204/204 |
| Pudou 754 | 274/274 | 209/209 | 324/324 | 137/137 | 252/252 | 179/179 | 306/306 | 129/129 | 201/201 |
| Heyu 10hao | 282/282 | 206/206 | 317/317 | 137/137 | 249/249 | 179/179 | 300/300 | 107/107 | 204/204 |
| Liudou 108 | 274/274 | 209/209 | 324/324 | 137/137 | 246/246 | 179/179 | 300/300 | 129/129 | 204/204 |
| Huaidou 17 | 274/274 | 209/209 | 324/324 | 137/137 | 249/249 | 179/179 | 306/306 | 129/129 | 204/204 |
| Nannong 60 | 274/274 | 200/200 | 314/314 | 150/150 | 246/246 | 179/179 | 300/300 | 107/107 | 201/201 |
| Shengyu 6hao | 274/274 | 209/209 | 324/324 | 137/137 | 252/252 | 179/179 | 300/300 | 148/148 | 204/204 |
| Xu 9416-8 | 274/274 | 200/200 | 324/324 | 131/131 | 243/252 | 179/179 | 312/312 | 129/129 | 204/204 |
| Hedou 37 | 274/274 | 206/206 | 302/302 | 137/137 | 246/246 | 182/182 | 300/300 | 107/107 | 201/201 |
| Zhongdou 48 | 274/274 | 188/188 | 333/333 | 131/131 | 249/249 | 179/179 | 306/306 | 123/123 | 204/204 |
| Zhongdou 66 | 265/265 | 209/209 | 330/330 | 140/140 | 243/243 | 179/179 | 300/300 | 123/123 | 204/204 |
| Xiangchun 2704 | 274/274 | 209/209 | 333/333 | 140/140 | 249/249 | 179/179 | 300/300 | 123/123 | 204/204 |
| Wandou 40 | 274/274 | 209/209 | 324/324 | 137/137 | 252/252 | 179/179 | 300/300 | 126/126 | 204/204 |
| Huadou 15 | 274/274 | 209/209 | 330/330 | 131/131 | 249/249 | 182/182 | 300/300 | 107/107 | 213/213 |
| Zhoudou 49 | 274/274 | 209/209 | 317/317 | 137/137 | 243/243 | 179/179 | 300/300 | 129/129 | 204/204 |
| Huadou 16 | 274/274 | 206/206 | 324/324 | 137/137 | 249/249 | 179/179 | 306/306 | 129/129 | 201/201 |
| Wandou 61 | 274/274 | 188/188 | 324/324 | 131/131 | 243/243 | 176/176 | 300/300 | 129/129 | 204/204 |
| Jiaoda 23 | 265/265 | 191/191 | 330/330 | 86/86 | 246/246 | 182/182 | 306/306 | 123/123 | 219/219 |
| Jiaoda 28 | 265/265 | 191/191 | 302/333 | 86/86 | 249/249 | 182/182 | 300/300 | 123/123 | 204/204 |
| Zhexian 86 | 274/274 | 209/209 | 324/324 | 86/86 | 249/249 | 182/182 | 306/306 | 107/107 | 213/213 |
| Huachun 12 | 274/274 | 188/188 | 330/330 | 140/140 | 249/249 | 182/182 | 312/312 | 126/126 | 204/204 |
| Sudou 051 | 265/265 | 197/197 | 336/336 | 131/131 | 249/249 | 179/179 | 306/306 | 107/107 | 204/204 |
| Zhoudou 38 | 265/265 | 188/188 | 324/324 | 150/150 | 249/249 | 179/179 | 300/300 | 110/110 | 204/204 |
| Zhongdou 6301 | 274/274 | 188/188 | 330/330 | 137/137 | 252/252 | 179/179 | 300/300 | 129/129 | 213/213 |
| Wansu 061 | 274/274 | 188/188 | 317/317 | 150/150 | 246/246 | 179/179 | 300/300 | 126/126 | 201/201 |
| Wansu 112 | 274/274 | 200/200 | 336/336 | 131/131 | 249/249 | 179/179 | 306/306 | 107/107 | 204/204 |
| Variety name | Gm022 | Gm027 | Gm029 | Gm032 | Gm033 | Gm043 | Gm045 | Gm046 | Gm048 |
| Xingnong 25 | 136/146 | 278/278 | 338/338 | 304/304 | 154/154 | 97/97 | 333/333 | 203/203 | 158/158 |
| Suinong 117 | 136/136 | 243/243 | 338/338 | 304/304 | 154/154 | 97/97 | 363/363 | 203/203 | 158/158 |
| Zhongnong 606 | 146/146 | 275/275 | 338/338 | 298/319 | 154/154 | 97/97 | 333/363 | 203/203 | 176/176 |
| Longken 397 | 146/146 | 278/278 | 338/338 | 304/304 | 154/154 | 97/97 | 333/333 | 203/203 | 158/158 |
| Jiyu 209 | 136/136 | 275/275 | 335/335 | 304/304 | 154/154 | 97/97 | 363/363 | 203/203 | 176/176 |
| Xingnong 9 | 146/146 | 278/278 | 338/338 | 304/304 | 154/154 | 97/97 | 333/333 | 203/216 | 158/167 |
| Henong 81 | 136/136 | 278/278 | 356/356 | 298/298 | 154/154 | 97/97 | 333/333 | 182/203 | 158/158 |
| Kendou 94 | 146/146 | 263/263 | 338/338 | 298/298 | 154/154 | 97/97 | 333/333 | 203/203 | 158/158 |
| Fenghedou 305 | 146/146 | 243/243 | 338/338 | 298/298 | 154/154 | 97/97 | 363/363 | 203/203 | 158/158 |
| Tiedou 102 | 146/146 | 263/263 | 335/335 | 319/319 | 134/134 | 97/97 | 333/336 | 182/203 | 176/176 |
| Liaodou 69 | 136/136 | 263/263 | 356/356 | 298/298 | 154/154 | 97/97 | 351/351 | 219/219 | 179/179 |
| Tiedou 110 | 146/146 | 233/275 | 338/338 | 304/304 | 134/134 | 97/97 | 354/354 | 203/203 | 173/173 |
| Shengdou 21 | 146/146 | 230/230 | 335/335 | 319/319 | 154/154 | 97/97 | 327/327 | 182/182 | 158/158 |
| Zhonghuang 203 | 146/146 | 230/230 | 335/335 | 304/304 | 154/154 | 97/97 | 333/333 | 219/219 | 170/170 |
| Zhonghuang 212 | 146/146 | 278/278 | 335/335 | 304/304 | 154/154 | 97/97 | 333/333 | 219/219 | 170/170 |
| Andou 6223 | 146/146 | 230/230 | 353/353 | 298/298 | 134/134 | 97/97 | 360/360 | 182/182 | 173/173 |
| Jidou 29 | 146/146 | 263/275 | 335/338 | 319/319 | 154/154 | 97/97 | 363/363 | 203/203 | 173/173 |
| Zhonghuang 211 | 146/146 | 230/230 | 335/335 | 304/304 | 154/154 | 97/97 | 333/333 | 219/219 | 170/170 |
| Ji 1901 | 146/146 | 275/275 | 335/335 | 307/307 | 157/157 | 97/97 | 333/333 | 216/216 | 179/179 |
| Andou 6263 | 146/146 | 230/278 | 338/338 | 304/304 | 134/134 | 85/85 | 327/327 | 219/219 | 173/173 |
| Handou 13 | 146/146 | 230/230 | 356/356 | 304/304 | 154/154 | 85/85 | 354/357 | 182/203 | 158/158 |
| Shanning 29 | 146/146 | 243/243 | 353/353 | 304/304 | 157/157 | 97/97 | 357/357 | 182/219 | 170/170 |
| Ji 1801 | 146/146 | 278/278 | 356/356 | 304/304 | 134/134 | 97/97 | 363/363 | 182/203 | 158/158 |
| Pudou 754 | 146/146 | 278/278 | 350/350 | 298/298 | 134/134 | 97/97 | 327/327 | 219/219 | 164/164 |
| Heyu 10hao | 146/146 | 230/243 | 353/353 | 307/307 | 154/154 | 97/97 | 333/333 | 219/219 | 164/164 |
| Liudou 108 | 146/146 | 230/230 | 353/353 | 304/304 | 154/154 | 85/85 | 354/354 | 182/182 | 158/158 |
| Huaidou 17 | 146/146 | 278/278 | 353/353 | 298/298 | 134/134 | 97/97 | 327/327 | 182/182 | 164/164 |
| Nannong 60 | 146/146 | 230/230 | 347/347 | 319/319 | 154/154 | 97/97 | 354/354 | 182/203 | 158/158 |
| Shengyu 6hao | 146/146 | 243/243 | 350/350 | 304/304 | 134/134 | 97/97 | 327/327 | 182/182 | 161/161 |
| Xu 9416-8 | 146/146 | 230/230 | 353/353 | 304/304 | 134/134 | 97/97 | 354/354 | 219/219 | 170/170 |
| Hedou 37 | 146/146 | 230/230 | 356/356 | 304/304 | 154/154 | 85/85 | 354/354 | 219/219 | 158/158 |
| Zhongdou 48 | 136/136 | 230/230 | 338/338 | 304/304 | 134/134 | 97/97 | 351/351 | 182/182 | 170/170 |
| Zhongdou 66 | 146/146 | 230/230 | 338/338 | 298/298 | 134/134 | 97/97 | 351/351 | 182/182 | 170/170 |
| Xiangchun 2704 | 136/136 | 278/278 | 335/335 | 298/298 | 134/134 | 97/97 | 351/351 | 182/182 | 170/170 |
| Wandou 40 | 146/146 | 278/278 | 350/350 | 298/298 | 134/134 | 97/97 | 354/354 | 182/182 | 164/164 |
| Huadou 15 | 146/146 | 230/230 | 356/356 | 298/298 | 134/134 | 97/97 | 333/333 | 207/207 | 170/170 |
| Zhoudou 49 | 146/146 | 275/275 | 341/341 | 307/307 | 134/134 | 97/97 | 354/354 | 182/182 | 164/164 |
| Huadou 16 | 146/146 | 230/230 | 353/353 | 304/304 | 154/154 | 85/97 | 360/360 | 219/219 | 173/173 |
| Wandou 61 | 146/146 | 243/243 | 353/353 | 307/307 | 157/157 | 97/97 | 354/354 | 182/182 | 164/164 |
| Jiaoda 23 | 136/146 | 243/243 | 338/338 | 304/304 | 154/154 | 97/97 | 342/342 | 203/203 | 173/173 |
| Jiaoda 28 | 136/136 | 243/243 | 356/356 | 304/304 | 154/154 | 97/97 | 342/354 | 203/203 | 173/173 |
| Zhexian 86 | 146/146 | 230/230 | 341/341 | 304/304 | 134/134 | 97/97 | 351/351 | 182/182 | 170/170 |
| Huachun 12 | 146/146 | 263/263 | 341/341 | 304/304 | 134/134 | 97/97 | 354/354 | 182/182 | 176/176 |
| Sudou 051 | 146/146 | 230/230 | 353/353 | 304/304 | 134/134 | 97/97 | 360/360 | 219/219 | 170/170 |
| Zhoudou 38 | 146/146 | 243/243 | 353/353 | 307/307 | 134/134 | 85/85 | 354/354 | 182/182 | 170/170 |
| Zhongdou 6301 | 146/146 | 230/278 | 356/356 | 298/298 | 134/134 | 97/97 | 333/333 | 182/203 | 170/170 |
| Wansu 061 | 146/146 | 230/243 | 338/338 | 304/304 | 134/134 | 85/85 | 354/354 | 182/203 | 158/158 |
| Wansu 112 | 146/146 | 230/263 | 335/335 | 304/304 | 134/134 | 97/97 | 360/360 | 219/219 | 170/170 |

| Variety name | Gm049 | Gm051 | Gm052 | Gm053 | Gm054 | Gm055 | Gm056 | Gm059 | Gm065 | Gm070 |
|---|---|---|---|---|---|---|---|---|---|---|
| Xingnong 25 | 216/216 | 144/144 | 373/373 | 351/351 | 267/267 | 147/147 | 274/274 | 272/272 | 300/300 | 326/326 |
| Suinong 117 | 216/216 | 132/132 | 373/373 | 351/351 | 260/260 | 147/147 | 274/274 | 298/298 | 300/300 | 326/326 |
| Zhongnong 606 | 237/237 | 132/141 | 364/364 | 342/342 | 267/267 | 147/147 | 274/274 | 275/298 | 300/300 | 326/326 |
| Longken 397 | 237/237 | 144/144 | 364/364 | 351/351 | 260/260 | 147/147 | 274/274 | 272/272 | 300/300 | 326/326 |
| Jiyu 209 | 237/237 | 147/147 | 364/364 | 328/328 | 245/245 | 147/147 | 271/271 | 298/298 | 300/300 | 326/326 |
| Xingnong 9 | 237/237 | 144/144 | 373/373 | 351/351 | 267/267 | 147/147 | 274/274 | 298/298 | 300/300 | 326/326 |
| Henong 81 | 216/216 | 144/144 | 373/373 | 351/351 | 267/267 | 147/147 | 274/274 | 272/272 | 300/300 | 326/326 |
| Kendou 94 | 237/237 | 144/144 | 364/364 | 351/351 | 267/267 | 128/128 | 271/271 | 272/272 | 300/300 | 326/326 |
| Fenghedou 305 | 216/216 | 141/141 | 373/373 | 351/351 | 267/267 | 147/147 | 274/274 | 275/275 | 300/300 | 326/326 |
| Tiedou 102 | 240/240 | 132/132 | 349/349 | 345/345 | 267/267 | 144/144 | 271/271 | 298/298 | 300/300 | 326/326 |
| Liaodou 69 | 237/237 | 132/132 | 349/349 | 328/328 | 267/267 | 134/134 | 271/271 | 298/298 | 300/300 | 326/326 |
| Tiedou 110 | 237/237 | 144/144 | 364/364 | 328/328 | 263/263 | 144/144 | 271/271 | 298/298 | 300/300 | 338/338 |
| Shengdou 21 | 240/240 | 138/138 | 376/376 | 348/348 | 273/273 | 147/147 | 268/268 | 298/298 | 294/294 | 326/326 |
| Zhonghuang 203 | 213/216 | 132/132 | 349/349 | 328/328 | 245/245 | 147/147 | 271/271 | 301/301 | 294/294 | 326/326 |
| Zhonghuang 212 | 216/216 | 132/132 | 373/373 | 345/345 | 245/245 | 147/147 | 271/271 | 301/301 | 294/294 | 326/326 |
| Andou 6223 | 216/216 | 132/132 | 376/376 | 348/348 | 267/267 | 128/128 | 274/274 | 301/301 | 306/306 | 326/326 |
| Jidou 29 | 240/240 | 138/138 | 349/374 | 342/348 | 267/267 | 128/128 | 271/271 | 298/298 | 300/300 | 338/338 |
| Zhonghuang 211 | 213/216 | 132/132 | 349/349 | 328/328 | 245/245 | 147/147 | 268/271 | 301/301 | 294/300 | 326/326 |
| Ji 1901 | 240/240 | 132/132 | 349/349 | 328/328 | 260/260 | 147/147 | 274/274 | 298/298 | 300/300 | 326/326 |
| Andou 6263 | 231/231 | 132/132 | 376/376 | 348/348 | 248/248 | 128/128 | 274/274 | 301/301 | 294/294 | 326/326 |
| Handou 13 | 231/231 | 166/166 | 376/376 | 345/345 | 248/248 | 150/150 | 277/277 | 275/275 | 294/294 | 326/326 |
| Shanning 29 | 240/240 | 132/132 | 373/373 | 328/328 | 270/270 | 134/134 | 268/268 | 292/292 | 294/294 | 326/326 |
| Ji 1801 | 216/216 | 166/166 | 373/373 | 328/328 | 267/267 | 128/128 | 274/274 | 298/298 | 294/294 | 326/326 |
| Pudou 754 | 231/231 | 132/132 | 376/376 | 348/348 | 270/270 | 128/128 | 274/274 | 301/301 | 297/297 | 326/326 |
| Heyu 10hao | 231/231 | 132/132 | 376/376 | 348/348 | 273/273 | 134/134 | 277/277 | 277/298 | 294/294 | 326/326 |
| Liudou 108 | 231/231 | 132/132 | 376/376 | 333/348 | 248/248 | 134/134 | 274/274 | 301/301 | 297/297 | 326/326 |
| Huaidou 17 | 240/240 | 132/132 | 376/376 | 348/348 | 273/273 | 128/128 | 271/271 | 301/301 | 297/297 | 326/326 |
| Nannong 60 | 243/243 | 138/138 | 373/373 | 333/333 | 248/248 | 147/147 | 271/271 | 298/298 | 294/294 | 326/326 |
| Shengyu 6hao | 231/247 | 144/144 | 376/376 | 328/328 | 270/270 | 130/150 | 271/271 | 277/277 | 297/297 | 326/326 |
| Xu 9416-8 | 216/216 | 132/144 | 376/376 | 348/348 | 245/245 | 134/134 | 277/277 | 277/277 | 294/294 | 326/326 |
| Hedou 37 | 231/231 | 166/166 | 376/376 | 333/333 | 248/248 | 134/134 | 277/277 | 298/298 | 294/294 | 326/326 |
| Zhongdou 48 | 222/222 | 132/132 | 373/373 | 336/336 | 245/245 | 128/128 | 268/271 | 272/272 | 294/294 | 338/338 |
| Zhongdou 66 | 231/231 | 163/163 | 373/373 | 336/336 | 245/245 | 134/134 | 271/271 | 272/272 | 294/294 | 326/326 |
| Xiangchun 2704 | 231/231 | 163/163 | 349/349 | 336/336 | 267/267 | 128/128 | 268/268 | 272/272 | 294/294 | 338/338 |
| Wandou 40 | 234/234 | 144/144 | 376/376 | 348/348 | 273/273 | 134/134 | 271/271 | 277/277 | 294/294 | 326/326 |
| Huadou 15 | 231/231 | 166/166 | 379/379 | 336/336 | 270/270 | 134/134 | 268/268 | 301/301 | 294/294 | 326/326 |
| Zhoudou 49 | 231/231 | 132/132 | 376/376 | 336/336 | 267/267 | 147/147 | 268/271 | 298/298 | 294/294 | 326/326 |
| Huadou 16 | 240/240 | 132/132 | 376/376 | 328/328 | 248/248 | 134/134 | 277/277 | 301/301 | 297/297 | 326/326 |
| Wandou 61 | 234/234 | 132/132 | 376/376 | 328/328 | 270/270 | 128/128 | 268/268 | 277/277 | 306/306 | 326/326 |
| Jiaoda 23 | 225/225 | 144/144 | 370/370 | 345/345 | 257/257 | 147/147 | 268/271 | 272/272 | 294/294 | 326/326 |
| Jiaoda 28 | 234/245 | 144/144 | 346/364 | 345/345 | 257/257 | 128/128 | 268/268 | 272/272 | 300/300 | 326/326 |
| Zhexian 86 | 231/246 | 144/144 | 373/373 | 345/345 | 248/248 | 134/134 | 271/271 | 301/301 | 294/294 | 326/326 |
| Huachun 12 | 237/237 | 166/166 | 373/373 | 348/348 | 245/245 | 144/144 | 274/274 | 298/298 | 294/294 | 326/326 |
| Sudou 051 | 216/216 | 132/132 | 376/376 | 333/333 | 270/270 | 134/134 | 268/268 | 301/301 | 294/294 | 326/326 |
| Zhoudou 38 | 213/213 | 144/144 | 373/373 | 348/348 | 267/267 | 147/147 | 268/268 | 298/298 | 306/306 | 326/326 |
| Zhongdou 6301 | 234/234 | 132/132 | 373/373 | 336/336 | 267/267 | 128/131 | 271/271 | 301/301 | 294/294 | 326/326 |
| Wansu 061 | 231/231 | 144/144 | 376/376 | 999/999 | 267/267 | 147/147 | 268/268 | 298/298 | 291/291 | 999/999 |
| Wansu 112 | 216/216 | 144/144 | 376/376 | 333/333 | 270/270 | 134/134 | 268/268 | 301/301 | 294/294 | 326/326 |

NOTE: all soybean varieties used were from the National Crop Germplasm Bank of China.

The results in Table 2 shows that compared with the traditional SSR marker detection, the SSR marker developed in the present disclosure could complete PCR amplification and capillary electrophoresis in one well with 9 to 10 primer pairs, greatly reducing the experimental cost. The simultaneous application of 3 sets of primers improved the efficiency of marker detection and variety identification. Different primers were labeled with different fluorescent groups, and a molecular weight of an amplified fragment at each SSR site for each soybean material could be obtained by the capillary electrophoresis.

The above description of the disclosed embodiments enables those skilled in the art to achieve or use the present disclosure. Various modifications to these embodiments are readily apparent to those skilled in the art, and the generic principles defined herein may be practiced in other embodiments without departing from the spirit or scope of the present disclosure. Thus, the present disclosure is not limited to the examples shown herein but falls within the widest scope consistent with the principles and novel features disclosed herein.

What is claimed is:

1. A method for high-throughput multiplex PCR in soybean using a simple sequence repeat (SSR) marker, comprising the following sequences of an SSR primer set:
primer pair 1:
TCACTTGACTAATCGTGTCATGACA set forth in SEQ ID NO: 1;
AGAATGGACTAACGTTGAGAGGATT set forth in SEQ ID NO: 2;
primer pair 2:
GCTAACCCGCTCTATGTTAAAGTTC set forth in SEQ ID NO: 3;
ATAGAGACAGAGCCAACTCAAACTT set forth in SEQ ID NO: 4;
primer pair 3:
AACACCAAACTATCACTAGGTTTGC set forth in SEQ ID NO: 5;
CCAGGTCACTTACACCTTAACACTA set forth in SEQ ID NO: 6;
primer pair 4:
GATGTCTGCCTCTATGGTAGCTTAA set forth in SEQ ID NO: 7;
AGTAGTTGTAAGCATACCACCATGA set forth in SEQ ID NO: 8;
primer pair 5:
GCAATCAATTTGTGTCTTTATGCCC set forth in SEQ ID NO: 9;
TTTTGAATCCTAGACATGCATGCAA set forth in SEQ ID NO: 10;
primer pair 6:
TCACCATTTAGATTCTTCTCAGGCA set forth in SEQ ID NO: 11;
TTCGTGAGCTATTGTTTTTCATCGT set forth in SEQ ID NO: 12;
primer pair 7:
AGTCATGTCAGCAATTCAGCTTATG set forth in SEQ ID NO: 13;
GTCTCCCTCTCTTTCATTTCTACGA set forth in SEQ ID NO: 14;
primer pair 8:
TCAAATTTTAACCTCAGAGGACAGG set forth in SEQ ID NO: 15;
TGACAATGAAGATAATGATAACAAAATTAGT set forth in SEQ ID NO: 16;
primer pair 9:
ATCATAGTCAGGGGTGGACCTATAT set forth in SEQ ID NO: 17;
AACAACATCATTCTGATCAGTGGTG set forth in SEQ ID NO: 18;
primer pair 10:
GACACCAATCAAAAATTGAACTGCA set forth in SEQ ID NO: 19;
ACTCAACCAATAAAAGAGCATGCAA set forth in SEQ ID NO: 20;
primer pair 11:
TCAAGCACTTTGATTTCCATTCTGT set forth in SEQ ID NO: 21;
CGTGTAGTATAGTTTTGAAAATGGCG set forth in SEQ ID NO: 22;
primer pair 12:
AAAAACCATCCTTACAAAACTGCCA set forth in SEQ ID NO: 23;
TCAGTGGGATCTGATTGTATTTTACC set forth in SEQ ID NO: 24;
primer pair 13:
AAAACATCTCTTCATGCTGTTGTCC set forth in SEQ ID NO: 25;
AAACCTAACTAAGCTTCGGTCTCAA set forth in SEQ ID NO: 26;
primer pair 14:
TGTATACACGAAAGATGGAATATTCTTTT set forth in SEQ ID NO: 27;
TGCCGGAAAATTTTATGGCATAGAT set forth in SEQ ID NO: 28;
primer pair 15:
TTAAGCAGTTCCTCTCATCACGTAA set forth in SEQ ID NO: 29;
TGGATAGTTGACTTTGTTTGGTTGG set forth in SEQ ID NO: 30;
primer pair 16:
AGTTGGTTAAGTCGATTAGGCTTGA set forth in SEQ ID NO: 31;
GCCAAAAATGAGCAAAAATGCAACA set forth in SEQ ID NO: 32;
primer pair 17:
GAGCTATTCCTTTGATTGAAACCCA set forth in SEQ ID NO: 33;
TTGGGAGCTTATACTGAGGTTTCTT set forth in SEQ ID NO: 34;
primer pair 18:
TGTTTAGTCAATTCAGGTCGATAAGT set forth in SEQ ID NO: 35;
GTTCACATGCTTGTACCGATTGATA set forth in SEQ ID NO: 36;
primer pair 19:
CCAGAGTTAACATCTCGGTTTGATG set forth in SEQ ID NO: 37;
AATTTGGCCAAATTCAAACTGGTCA set forth in SEQ ID NO: 38;
primer pair 20:
TTTTAGCCAAAGAACATGTTATGGT set forth in SEQ ID NO: 39;
ACTTTTATTCAACATGCTTTTCATAAGT set forth in SEQ ID NO: 40;
primer pair 21:
CGCCCACCAATAGAAATAATTGTGA set forth in SEQ ID NO: 41;
TGTGATCGGATGTTAATTAGCTTTTTCA set forth in SEQ ID NO: 42;
primer pair 22:
TATCCATCGTGTTGCTGTCATACTT set forth in SEQ ID NO: 43;
ACCATTGTCCTTATCATATTCGTTAAAAA set forth in SEQ ID NO: 44;
primer pair 23:
TAATTCTAGCTGGCCTTTAGAACGT set forth in SEQ ID NO: 45;
ATATTTAAGTGGCGTTGGATTCGAC set forth in SEQ ID NO: 46;
primer pair 24:
GCACACATCATTTCTTTGTTTGACC set forth in SEQ ID NO: 47;
TGTGTTCCGATTTTGATTGCGATAA set forth in SEQ ID NO: 48;
primer pair 25:
TCTTTGTAAGATCACGCCATTATTT set forth in SEQ ID NO: 49;
GTTGCATTGCACATTAGGTTTTCTG set forth in SEQ ID NO: 50;
primer pair 26:
ACAAAAATCAACAACGTTAACAATATAAATT set forth in SEQ ID NO: 51;
GGCAGGGTGGAAGTGAATTTTT set forth in SEQ ID NO: 52;
primer pair 27:
TACGTATTTTCATTGCACAAGTTGT set forth in SEQ ID NO: 53;
TGTCTATGTTCTACCATTAAATGATGACA set forth in SEQ ID NO: 54;
primer pair 28:
ATCCCCAAAGTTATGGAAGAGTCAA set forth in SEQ ID NO: 55;
TCAAGAAACAAAAGGTATGCATGCT set forth in SEQ ID NO: 56.

2. The method according to claim 1, comprising the following steps:
(1) extracting DNA from a soybean sample;
(2) establishing a PCR reaction system using the DNA as a template and the SSR primer set according to claim 1 as an amplification primer and conducting PCR amplification; and
(3) subjecting an obtained amplified product to capillary electrophoresis, and then detecting a length of an obtained fragment.

3. The method according to claim 2, wherein the PCR reaction system for the PCR amplification is 25 μL in volume and comprises 1.5 μL of primer mixture, 5 μL of DNA, 12.5 μL of Premix Ex Taq Hot Start enzyme, and 6 μL of water;
a single primer in the primer mixture has a concentration of 0.4 μM, and the DNA has a concentration of 20 ng/μL; and
a procedure of the PCR amplification comprises: initial denaturation at 95° C. for 3 min; 32 cycles for a process of denaturation at 95° C. for 30 s and then annealing at 60° C. for 4 min; and extension at 72° C. for 5 min.

4. The method according to claim 1, wherein a soybean variety to be identified comprises:
Xingnong 25, Suinong 117, Zhonglong 606, Longken 397, Jiyu 209, Xingnong 9, Henong 81, Kendou 94, Fenghedou 305, Tiedou 102, Liaodou 69, Tiedou 110, Shengdou 21, Zhonghuang 203, Zhonghuang 212, Andou 6223, Jidou 29, Zhonghuang 211, Ji 1901, Andou 6263, Handou 13, Shanning 29, Ji 1801, Pudou 754. Heyu 10hao, Liudou 108, Huaidou 17, Nannong 60, Shengyu 6hao, Xu 9416-8, Hedou 37, Zhongdou 48, Zhongdou 66, Xiangchun 2704, Wandou 40, Huadou 15, Zhoudou 49, Huadou 16, Wandou 61, Jiaoda 23, Jiaoda 28, Zhejiangxian 86, Huachun 12, Sudou 051, Zhoudou 38, Zhongdou 6301, Wansu 061, and Wansu 112.
